(19) United States
(12) Patent Application Publication
(10) Pub. No.: US 2006/0095134 A1
(43) Pub. Date: May 4, 2006

(54) Materials, devices and methods for implantation of transformable implants

(75) Inventors: Hai H. Trieu, Cordova, TN (US); Michael C. Sherman, Memphis, TN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SDGI Holdings, Inc.

(21) Appl. No.: 10/975,049

(22) Filed: Oct. 28, 2004

(51) Int. Cl. A61F 2/44; A61B 17/70

(52) U.S. Cl. 623/17.16; 606/61; 606/76; 623/13.11

(57) ABSTRACT

A transformable implantable device is disclosed comprising primary and secondary phases or materials. The secondary phase or material is relatively rigid compared to the primary phase or material and also renders the transformable implantable device relatively rigid compared to the primary phase or material. The secondary phase or material, upon implantation, becomes more flexible, thereby rendering the transformable implantable device more flexible also.

### FIELD OF THE INVENTION

The present invention relates generally to implantable devices and more specifically to implantable devices that are initially rigid but become flexible after implantation.

### BACKGROUND OF THE INVENTION

Implantable devices are used to rectify a variety of medical ailments. For example, implants often are used to treat disorders in the vertebral column. The vertebral column (spine) is a biomechanical structure composed primarily of ligaments, muscles, vertebrae and intervertebral discs. The biomechanical functions of the spine include (i) support of the body; (ii) regulation of the motion between the head, trunk, arms, pelvis, and legs; and (iii) protection of the spinal cord and the nerve roots.

Damage to one or more components of the vertebral column, such as an intervertebral disc, may result from disease or trauma and cause instability of a portion or all of the vertebral column. A common treatment for a damaged vertebral column is spinal fixation or fusion wherein some or all of the intervertebral joints are permanently fixed. Intervertebral joints consist of two adjacent vertebrae and their posterior bony elements connected by an intervertebral disc, ligaments, and two facet joint capsules. Spinal fusion is sometimes accomplished using bone grafts to fuse the adjacent vertebrae. Fusion also may involve the insertion of intervertebral disc devices and surgical procedures on the disc space or vertebral bodies.

Additionally, a spinal fixation device may be installed to stabilize the spinal column and promote the fusion of intervertebral joints. A rigid spinal fixation device consists of a rigid stabilizing element, such as rods or plates, attached by anchors to the vertebrae in the section of the vertebral column that is to be fused. For example, a rigid metal plate can be placed along the anterior aspect of the vertebrae and secured to the vertebrae using titanium screws. The spinal fixation device restricts the movement of the fused vertebrae relative to one another and supports all or part of the stresses imparted to the vertebral column instead of the series of vertebrae and intervertebral joints across which the implant spans.

However, there are some disadvantages associated with the use of rigid spinal fixation devices. For example, fixing a series of vertebrae may localize stress at the intervertebral discs located at either end of the series of fixed vertebrae and can lead to abnormal degeneration of these disks. Additionally, the attachment points of the anchors of the rigid spinal fixation device are subject to significant forces that may cause loosening of the anchors and damage to the vertebrae in which the anchors are secured. Also, the rerouting of stresses around vertebrae by the rigid spinal fixation device may lead to bone loss because of the decreased load upon the vertebrae; this effect is called stress shielding. Another drawback of rigid spinal fixation devices is the intrusion of the rigid device into the adjacent tissue and vasculature, causing damage and discomfort. Yet another disadvantage is the reduced mobility caused by the fusion of the intervertebral joints.

In response, flexible spinal fixation devices have been employed. These devices are designed to partially or fully support the stresses imparted to the vertebral column but also allow a degree of movement that absorbs some of the stresses placed on the vertebral column rather than transferring the stresses to the attachment points and adjacent free vertebrae. In this way, flexible spinal fixation devices avoid some of the disadvantages of rigid spinal fixation devices.

For example, U.S. Pat. No. 6,652,585, the disclosure of which is incorporated herein in its entirety, describes a flexible spine stabilization system designed to replace the anterior longitudinal ligament. The device is a flexible metal or polymer plate attached to the anterior portion of the vertebrae that resists extension and rotation of the spine but does not aid in absorbing the compressive loading of the spine.

U.S. Pat. No. 5,282,863, the disclosure of which also is incorporated herein in its entirety, describes a flexible stabilization system for a vertebral column. The stabilization system consists of anchoring means and a porous stabilization element modeled after sea coral.

U.S. Re. Pat. No. 36,221, the disclosure of which is incorporated herein in its entirety, describes a flexible inter-vertebral stabilizer. The stabilizer is a supple band made of a flexible plastic material having all-directional flexibility.

U.S. Pat. App. No. 2002/0123750, the disclosure of which is incorporated herein in its entirety, describes a woven orthopedic implant. The implant is made from a mesh material that may be treated in order to promote bone growth or provide other special benefits. The mesh may be used as a prosthetic ligament, a tension band, or a fixation device.

U.S. Pat. No. 5,415,661, the disclosure of which is incorporated herein in its entirety, describes an implantable spinal assist device. The device is a curvilinear body composed of a composite material made up of a carbon or polyamide fiber dispersed in a biocompatible polymer matrix.

Flexible implantable devices also are used in the treatment of damaged or diseased intervertebral discs. The intervertebral disc functions to stabilize the spine and to distribute forces between vertebral bodies. The intervertebral disc is composed of three structures: the nucleus pulposus, the annulus fibrosis, and two vertebral end-plates. These components work to absorb the shock, stress, and motion imparted to the human vertebrae. The nucleus pulposus is an amorphous hydrogel with the capacity to bind water. It is maintained within the center of an intervertebral disc by the annulus fibrosis, which is composed of highly structured collagen fibers. The vertebral end-plates, composed of hyalin cartilage, separate the disc from adjacent vertebral bodies and act as a transition zone between the hard vertebral bodies and the soft disc.

Like other components of the vertebral column, intervertebral discs also may be damaged by trauma or disease, leading to reduced disc space height, instability of the spine, decreased mobility, and pain. One way to treat a damaged intervertebral disc is by surgical removal of a portion or all of the intervertebral disc. The removal of the damaged or unhealthy disc may allow the disc space to collapse, which would lead to instability of the spine, abnormal joint mechanics, nerve damage, and severe pain. Therefore, prosthetic intervertebral disc implant devices may be used to replace the removed portion of the natural intervertebral disc. These prosthetic discs are sufficiently flexible to absorb the compressive forces of the spine on the intervertebral disc and allow for rotational movement of the spine.

U.S. Pat. No. 6,264,695, the disclosure of which is incorporated herein in its entirety, describes an intervertebral disc nucleus implant. The implant is a composite device with a cellular matrix and a hydrophilic phase, such as a hydrogel. The cellular matrix supports the compressive load placed on the implant and the hydrophilic phase expands the implant following implantation. In this way, the implant may be dehydrated prior to implantation to facilitate insertion through a small defect or hole in the annulus but will expand to fill the evacuated disc space when the implant is contacted by bodily fluids.

U.S. Pat. No. 5,976,186, the disclosure of which is incorporated herein in its entirety, describes a hydrogel intervertebral disc nucleus. The hydrogel nucleus is dehydrated, shaped to form a rod or tube, and inserted into the evacuated disc space. The hydrogel expands to fill the evacuated disc space upon contact with bodily fluids.

U.S. Pat. No. 5,458,643, the disclosure of which is incorporated herein in its entirety, describes an artificial intervertebral disc. The artificial disk comprises a layer of polyvinyl alcohol hydrogel positioned between layers of titanium mesh or alumina ceramic. The hydrogel layer provides extra cushioning to the intervertebral implant.

Flexible implants, such as spinal fixation devices and intervertebral discs, however, possess some disadvantages. Flexible implants are more easily deformed or deflected by surrounding tissues during implantation, making surgical installation of the implants more difficult. Particularly where minimally invasive surgical techniques such as laparoscopic surgery are used, flexible implants may be difficult to install because the flexible materials may not be easily inserted through laparoscopic probes and other such devices. Additionally, flexible devices may not offer sufficient support to the damaged area or structure of the body, especially during initial healing of the area or structure.

The description herein of problems and disadvantages of known apparatus, methods, and devices is not intended to limit the invention to the exclusion of these known entities. Indeed, embodiments of the invention may include one or more of the known apparatus, methods, and devices without suffering from the disadvantages and problems noted herein.

### SUMMARY OF THE INVENTION

An improved flexible implant, including an improved flexible spinal fixation device, flexible intervertebral disc implant, and anterior spinal tension band would be advantageous. A number of advantages associated with the present invention are readily evident to those skilled in the art, including economy of design and resources, ease of use, quality of final product, cost savings, etc.

It therefore is a feature of an embodiment of the present invention to provide a transformable implantable device that is transformable from a relatively rigid state facilitating implantation, to a relatively flexible state. The invention may have one or more relatively flexible primary phases or materials. A secondary phase or material makes the implant relatively rigid until it is contacted by sufficient amounts of water or fluid. Thereafter, the implant returns to the relatively flexible state of the primary phases or materials.

Still further features and advantages of the present invention are identified in the ensuing description, with reference to the drawings identified below.

### BRIEF DESCRIPTION OF THE DRAWINGS

FIG. 1, embodiments a and b are drawings of exemplary configurations of the invention as plates for use in spinal fixation devices.

FIG. 2 is a drawing of an exemplary configuration of the invention as an intervertebral disc implant.

FIGS. 3 and 4 are drawings of exemplary configurations of the invention as spinal fixation devices.

FIG. 5 is a drawing of an exemplary configuration of the invention as an intervertebral disc implant.

FIG. 6 is a drawing of an exemplary configuration of the invention as a spinal ligament repair and reinforcement device.

FIG. 7 is a drawing of an exemplary installation of the invention configured as spinal fixation plates.

### DETAILED DESCRIPTION OF THE INVENTION

The following description is intended to convey a thorough understanding of the present invention by providing a number of specific embodiments and details involving transformable implantable devices. It is understood, however, that the present invention is not limited to these specific embodiments and details, which are exemplary only. It is further understood that one possessing ordinary skill in the art, in light of known systems and methods, would appreciate the use of the invention for its intended purposes and benefits in any number of alternative embodiments.

In one exemplary embodiment, an implantable device is provided comprising at least one primary phase or material and a secondary phase or material. The secondary phase or material is relatively rigid compared to the primary phase or material. The secondary phase or material renders the implantable device as a whole relatively rigid, when compared to the primary phase or material. Upon implantation into the body, the secondary phase or material may be contacted by water or body fluids, however, and become more flexible. The implantable device thereby also becomes more flexible.

Any biocompatible, relatively flexible material may be used for the primary phase or material of the transformable implantable device. For example, metals, polymers, ceramics, shape memory alloys, and composites and mixtures thereof all may be appropriate for fabrication of the primary phase or material of the transformable implantable device.

Metals appropriate for the primary phase or material include, but are not limited to, stainless steel, particularly 316L stainless steel; cobalt-chrome alloys, cobalt-nickel-chrome alloys, particularly MP35N; titanium; titanium alloys, particularly Ti-6A1-4V; nickel-titanium shape memory alloys; composites, mixtures and alloys thereof; and the myriad different grades of these metals. The metal may be anodized, heat treated, cold forged, or otherwise treated prior to inclusion in the transformable implant for purposes of increasing the implant's strength, biocompatibility, or for other advantageous benefits.

Polymers appropriate for the primary phase or material of the transformable implantable device may be natural, semi-synthetic, or synthetic in on gin. Appropriate polymeric materials include, but are not limited to, biocompatible thermosetting polymers, thermoplastic polymers, elastomers, and mixtures thereof.

Natural biocompatible polymers, for example, may be chosen from the group consisting of biological adhesives such as fibrinogen, thrombin, mussel adhesive protein, casein, chitin or chitosan, natural or modified polysaccharides, polyethylene glycol derivatives, and starches.

Semi-synthetic biocompatible polymers include, but are not limited to, genetically-engineered protein polymers such as silk-like protein, and collagen-like protein.

Synthetic bio-compatible polymers include, for example, polyethylene, polyethylene terephthalate, polyvinyl alcohol, polypropylene, nylon, polyaryletherketone, polyacrylonitriles, expanded teflon (GORTEX®), as well as cyanoacrylates, epoxy-based compounds, gelatin-resorcinol-formaldehyde glues, polyacrylate, polymethyl methacrylate, polytetrafluoroethylene polyesters, and polyamides, particularly aromatic polyamides such as poly(paraphenylene terephthalamide). Other examples of synthetic polymers include, but are not limited to, polycarbonates including amino acid-derived polycarbonates, amino acid-derived polyarylates, polyarylates derived from dicarboxylic acids and amino acid-derived diphenols, anionic polymers derived from L-tyrosine, polyarylate random block copolymers, poly(hydroxycarboxylic acids), poly(caprolactones), poly(hydroxybutyrates), polyanhydrides, poly(orthoesters), polyesters, bisphenol-A based poly(phosphoesters), and polycyanoacrylates. Still other non-limiting examples of synthetic polymers include porous high density polyethylenes, polypropylenes, polyphenylenesulfides, polyacetals, polyamideimides, thermoplastic polyimides, polyaryletherketones, polyarylethernitriles, aromatic polyhydroxy-ethers, polyacrylonitriles, polyphenyleneoxides, polyesterurethane, polyester/polyol block copolymers, poly ethylene terepthalate, nylons, polysulphanes, polyaramids, polyvinyl chlorides, styrenic resins, polypropylenes, acrylonitrile-butadiene-styrene (“ABS”), acrylics, styrene acrylonitriles, and mixtures, copolymers, block polymers, and combinations thereof.

Suitable synthetic elastomers include polyurethane, silicone, copolymers of silicone and polyurethane, polyolefins such as polyisobutylene and polyisoprene, neoprene, nitrile, vulcanized rubber, and combinations thereof. Suitable thermoplastic polymers include, in particular, polysulfone and polyetherketone.

Those knowledgeable in the art will recognize the many different metals, polymers, ceramics, shape memory alloys, and composites thereof that may be appropriate for fabrication of the primary phase or material of the transformable implantable device. Suitable ceramic or inorganic materials include carbon fibers, boron fibers, and the like.

The primary phase of the transformable implantable device may be in any one of numerous physical forms including, but not limited to, a mesh, cellular matrix, fiber, sponge, or any other suitable form for use in the implantable device. Particularly for primary materials such as metals that are often rigid in bulk form, the form of the primary phase may be chosen to ensure that the primary phase or material is relatively flexible. For example, the primary material may be pulled into a fiber and then braided into a tether, cord, band, tubing, or otherwise. If the primary phase or material is in a fiber form, the fibers may be layered and directionally oriented. The directional orientation may be the same or different between layers. For example, layers of the primary phase fibers may be oriented perpendicular to one another in order to produce isotropic properties in the implant. Alternatively, layers of the primary phase polymers may be oriented in the same direction to produce anisotropic properties, particularly increased strength in the directions perpendicular to the orientation of the fibers.

Other variables also may be adjusted in order to ensure that the primary phase or material is relatively flexible compared to the secondary phase or material. For example, the fibers' diameters and lengths may be adjusted to ensure relative flexibility of the primary phase or material.

Any bio-compatible, hydrophilic material may be used for as the secondary phase or material of the transformable implantable device. In a preferred embodiment, the secondary phase or material is a hydrophilic polymer or hydrogel. Examples of synthetic hydrophilic polymers and hydrogels include, but are not limited to, polyethylene oxide, polyethylene glycol, polyvinyl alcohol, polyelectrolytes, polyacrylic acid, polyacrylamides such as poly(acrylonitrile-acrylic acid), and mixtures thereof. Examples of natural hydrophilic polymers and hydrogels include, but are not limited to, cellulosics such as ethyl cellulose, methyl cellulose, carboxy methyl cellulose, hydroxy ethyl cellulose, sodium carboxy methyl cellulose, and hydroxy propyl cellulose; collagen; gelatin; elastin; silk; keratin; and albumin. Additionally, copolymers such as silk and elastin copolymers and copolymers of polyacrylic acid and polyacrylamide are appropriate for use as the secondary phase or material. Also, blends such as cellulose with gelatin, composites such as polyvinyl alcohol and collagen, and laminates of multiple layers of hydrophilic polymers and hydrogels also are appropriate for use as the secondary phase or material.

Other exemplary hydrogels include those formed from polyacrylates such as poly(2-hydroxy ethyl methacrylate), copolymers of acrylates with N-vinyl pyrrolidone, N-vinyl lactams, polyurethanes, polyacrylonitrile, and other similar materials that form a hydrogel. Suitable natural hydrophilic polymer materials such as glucomannan gel, hyaluronic acid, polysaccharides such as cross-linked carboxyl-containing polysaccharides, collagen, elastin, albumin, keratin, and combinations thereof may be used as the secondary phase or material.

If desired, the polymers also may be cross-linked. Because cross-linking the polymers will increase the rigidity of the secondary phase, cross-linking may be used to adjust the flexibility of the implantable device. In a preferred embodiment, the secondary phase or material is bio-resorbable so that it will be removed from the body following implantation. Hydophilic ceramics such as calcium sulfate and calcium phosphate also may be used in the present invention. Those knowledgeable in the art will recognize the many different polymers, ceramics, and composites thereof that may be appropriate for use as the secondary phase or material of the transformable implantable device, using the guidelines provided herein.

The secondary phase or material may be added to or mixed with the primary phase or material at any appropriate time during manufacturing of the transformable implantable device. For example, if the device lends itself to manufacturing through extrusion, injection molding, compression molding, or solution casting processes, these processes can be carried out using mixtures of the primary and secondary phases or materials. Laminating and bonding processes can be carried out to produce the transformable implantable device using layers of primary and secondary phases or materials. If dispersion brushing, spraying, or dipping is appropriate for fabrication of the transformable implantable device, then the solution that is brushed, sprayed, or dipped may be a mixture in solution of the primary and secondary phases or materials. If necessary, additional machining, polishing, cutting, or other shaping steps may be performed on the device.

The transformable implantable device may contain additional agents or additives. The agents or additives may be mixed with the primary and secondary phases or materials during fabrication of the transformable implant. Alternatively, the transformable implant may be coated with an agent or additive, for example by powder coating, spraying, roller coating, dipping, etc. In another example, the transformable implant is immersed in a solution of the agents or additives whereby the agent or additive is incorporated into the implant. One skilled in the art will appreciate the various methods that may be employed to incorporate the agents or additives into the transformable implant. The additional agents or additives may be either in purified form, partially purified form, recombinant form, or any other form appropriate for inclusion in the transformable implant. It is preferred that the agent or additive be free of impurities and contaminants.

Many different additional agents or additives may be beneficially incorporated into the implant. For example, the transformable implant may be coated or impregnated with anti-adhesive material that will prevent tissue and vasculature from attaching to the implant. If a non-biocompatible substance is desired to be used as a primary or secondary phase or material, the non-biocompatible substance may be coated with a biocompatible polymer, ceramic, or metal to render it safe for use inside the body. Still other possible additional agents or additives that can be added to the implantable device include antibiotics, antiretroviral drugs, growth factors, fibrin, bone morphogenetic factors, bone growth agents, chemotherapeutics, pain killers, bisphosphonates, strontium salt, fluoride salt, magnesium salt, and sodium salt.

As stated above, the transformable implant further may comprise therapeutics, such as a pharmacological agent or biological agent. Examples of pharmacological agents or biological agents include, but are not limited to, antibiotics, analgesics, anti-inflammatory drugs, steroids, anti-viral and anti-retroviral compounds, therapeutic proteins or peptides, and therapeutic nucleic acids (as naked plasmid or a component of an integrating or non-integrating gene therapy vector system).

Antibiotics useful with the transformable implant include, but are not limited to, aminoglycosides, amoxicillin, ampicillin, azactam, bacitracin, beta-lactamases, beta-lactam (glycopeptide), biomycin, cefazolin, cephalosporins, ciprofloxacin, clindamycin, chloramphenicol, chloromycetin, erythromycin, fluoroquinolones, gentamicin, macrolides, metronidazole, peilicillins, polymycin B, quinolones, rapamycin, rifampin, streptomycin, sulfonamide, tetracyclines, tobramycin, trimethoprim, trimethoprim-sulfamthoxazole, vancomycin, and mixtures thereof. In addition, one skilled in the art of implant surgery or administrators of locations in which implant surgery occurs may prefer the introduction of one or more of the above-recited antibiotics to account for nosocomial infections or other factors specific to the location where the surgery is conducted. Accordingly, the invention further contemplates that one or more of the antibiotics, and any combination of one or more of the same antibiotics, may be included in the transformable implants of the invention.

The invention further contemplates that immunosuppressives may be administered with the transformable implants. Suitable immunosuppressive agents that may be administered in combination with the transformable implants include, but are not limited to, steroids, cyclosporine, cyclosporine analogs, cyclophosphamide, methylprednisone, prednisone, azathioprine, FK-506, 15-deoxyspergualin, and other immunosuppressive agents that act by suppressing the function of responding T cells. Other immunosuppressive agents that may be administered in combination with the transformable implants include, but are not limited to, prednisolone, methotrexate, thalidomide, methoxsalen, rapamycin, leflunomide, mizoribine (bredinin™), brequinar, deoxyspergualin, and azaspirane (SKF 105685), Orthoclone OKT™ 3 (muromonab-CD3). Sandimmune®, Neoral®, Sangdya® (cyclosporine), Prograf® (FK506, tacrolimus), Cellcept® (mycophenolate motefil, of which the active metabolite is mycophenolic acid), Imuran® (azathioprine), glucocorticosteroids, adrenocortical steroids such as Deltasone® (prednisone) and Hydeltrasol® (prednisolone), Folex® and Mexate® (methotrxate), Oxsoralen-Ultra® (methoxsalen) and Rapamuen® (sirolimus).

The invention also contemplates the use of therapeutic polynucleotides or polypeptides (hereinafter “therapeutics”) with the transformable implants of the invention. The therapeutics may be administered as proteins or peptides, or therapeutic nucleic acids, and may be administered as full length proteins, mature forms thereof or domains thereof, as well as the polynucleotides encoding the same. Examples of therapeutic polypeptides include, but are not limited to, demineralized bone matrix (DBM); bone morphogenetic proteins (BMPs), including BMP-1, BMP-2, BMP-3, BMP-4, BMP-5, BMP-6, BMP-7, BMP-8, BMP-9, BMP-10, BMP-11, BMP-12, BMP-13, BMP-15, BMP-16, BMP-17, and BMP-18; vascular endothelial growth factors (VEGFs), including VEGF-A, VEGF-B, VEGF-C, VEGF-D and VEGF-E; connective tissue growth factors (CTGFs), including CTGF-1, CTGF-2, and CTGF-3; osteoprotegerin; transforming growth factor betas (TGF-bs), including TGF-b-1, TGF-b-2, and TGF-b-3; platelet derived growth factors (PDGFs), including PDGF-A, PDGF-B, PDGF-C, and PDGF-D; insulin-related growth factor (IGF-I, IGF-II); fibroblast growth factor (FGF, bFGF, etc.); beta-2-microglobulin (BDGF II); fibronectin (FN); osteonectin (ON); endothelial cell growth factor (ECGF); cementum attachment extracts (CAE); ketanserin; human growth hormone (HGH); animal growth hormones; epidermal growth factor (EGF); interleukin-1 (IL-1); human alpha thrombin; and mixtures and combinations thereof. In addition, bone adhesives such as calcium phosphate, polymethacrylate and the like can be included in the transformable implant. The polynucleotides encoding the same also may be administered as gene therapy agents.

In a particularly preferred embodiment of the invention, the transformable implant comprises antagonists to either the myelin-associated glycoprotein (MAG) or Nogo-A, the largest transcript of the recently identified nogo gene (formerly called NI-220), which are both present in CNS myelin and have been characterized as potent inhibitors of axonal growth. For example, Nogo-A acts as a potent neurite growth inhibitor in vitro and represses axonal regeneration and structural plasticity in the adult mammalian CNS in vivo. In another embodiment of the invention, antagonists to both MAG and Nogo-A are co-administered to the patient. In this preferred embodiment of the invention, the transformable implants of the invention are used as implants for intervertebral discs that are adjacent to locations of spinal cord injury, and may also replace damaged or infected endogenous nucleus pulposus. In this embodiment of the invention, the inhibitory activity of the antagonist(s) to the activity of MAG and Nogo-A may aid in the regeneration of damaged spinal nerve tissue, and the transformable implant serves as a local reservoir of therapeutic antagonist(s) to aid in the growth of damaged spinal tissue. Antagonists of MAG and Nogo-A may take the form of monoclonal antibodies, anti-sense molecules, small molecule antagonists, and any other forms of protein antagonists known to those of skill in the art.

In this embodiment, therapeutic polypeptides or polynucleotides of Ninjurin-1 and Ninjurin-2 may further be administered alone or in conjunction with one or more MAG or Nogo-A antagonists, as a component of the transformable implant. Ninjurin-1 and Ninjurin-2 are believed to promote neurite outgrowth from primary cultured dorsal root ganglion neurons. Ninjurin-1 is a gene that is up-regulated after nerve injury both in dorsal root ganglion (DRG) neurons and in Schwann cells. The full-length proteins, mature forms, or domains of the full-length proteins thereof may be administered as therapeutics, as well as the polynucleotides encoding the same.

Still other growth agents include nucleic acid sequences that encode an amino acid sequence or an amino acid sequence itself wherein the amino acid sequence facilitates tissue growth or healing. For example, leptin is known to inhibit bone formation. Any nucleic acid or amino acid sequence that negatively impacts leptin, a leptin ortholog, or a leptin receptor may be included in the transformable implant in order to encourage bone growth, using the guidelines provided herein.

Other additional agents or additives that may be included in the transformable implant are chemotherapeutics such as cis-platinum, ifosfamide, methotrexate and doxorubicin hydrochloride. Those skilled in the art are capable of determining other chemotherapeutics that would be suitable for use in the implant.

The agent or additive also may be a pain killer or anti-inflammatory, such as non-steroidal anti-inflammatory drugs (NSAID). Examples of pain killers appropriate for inclusion in the transformable implant include, but are not limited to, lidocaine hydrochloride, bipivacaine hydrochloride, ibuprofren, and NSAIDs such as ketorolac tromethamine.

Still other examples of agents and additives that may be included in the transformable implant are biocidal/biostatic sugars such as dextran and glucose; peptides; vitamins; inorganic elements; co-factors for protein synthesis; hormones; endocrine tissue or tissue fragments; synthesizers; enzymes such as collagenase, peptidases, and oxidases; polymer cell scaffolds with parenchymal cells; angiogenic agents; antigenic agents; cytoskeletal agents; cartilage fragments; living cells such as chondrocytes, bone marrow cells, mesenchymal stem cells, natural extracts, genetically engineered living cells, or otherwise modified living cells; autogenous tissues such as blood, serum, soft tissue, and bone marrow; bioadhesives; periodontal ligament chemotactic factor (PDLGF); somatotropin; bone digestors; antitumor agents; immuno-suppressants; and permeation enhancers such as fatty acid esters including laureate, myristate, and stearate monoesters of polyethylene glycol.

A reinforcing component or structure such as a fiber, fibrous web, woven textile, nonwoven textile, mesh, nonflexible structural member or semiflexible structure member made from a natural, synthetic, or semisynthetic material, or combinations thereof, also may be added to the transformable implantable device. The reinforcing component may be useful to strengthen the transformable device and adjusting the flexibility of the device. Materials suitable for constructing the reinforcing component include, but are not limited to, collagen, tendons, keratin, cellulosics, ceramics, glass, metals and metal alloys, nylon fibers, carbon fibers, polyethylene fibers, and calcium phosphates. The reinforcing component can be nonbioresorbable or bioresorbable. Where practicable, it generally may be advantageous to orient the reinforcing component or structure along the axis of the forces that can be expected to be exerted against the transformable implant following its installation in the body.

After fabrication of the transformable implantable device, the secondary phase or material is substantially dehydrated. By “substantially dehydrated,” it is meant that at least 80%, more preferably at least 90%, and most preferably at least 95% of the water content of the secondary phase is removed. The substantial dehydration may, for example, be effected by freeze-drying, heating, air-drying, vacuum-heating, or vacuum-drying the device. In another exemplary method of substantially dehydrating the device, the device is rinsed with a solvent to displace the water from the secondary phase and the residual solvent then is removed from the implant by freeze-drying, heating, air-drying, vacuum-heating, vacuum-drying, or otherwise. When the secondary phase or material is substantially dehydrated, the transformable implantable device becomes relatively rigid.

Upon implantation of the transformable device in the body, however, the device is substantially re-hydrated by body fluids with which it comes into contact. By substantially re-hydrated, it is meant that the transformable device preferably absorbs at least 50%, more preferably at least 75%, and most preferably at least 95% of its capacity to hold water. The re-hydration of the device may take, for example, several minutes, hours, days, or weeks to complete. The temporal period of re-hydration may be adjusted, for example, by adjusting the permeability to body fluids of the transformable implant. As the re-hydration of the implant proceeds, the secondary phase or material will exert less of a stiffening effect on the implant, thereby allowing the implant to resume the relatively flexible state of the primary phase or material. Also, initial contact of the implant with body fluids upon insertion into the body may cause the re-hydration of the surface of the implant to provide a lubricating effect. This lubricating effect may aid in insertion of the transformable implant, especially where minimally-invasive surgical techniques such as laparoscopic surgery are utilized.

In another exemplary embodiment, a transformable implantable device in the form of a transformable spinal fixation device is provided. The transformable spinal fixation device comprises at least one primary phase or material and a secondary phase or material that is relatively rigid compared with the primary phase or material. The secondary phase or material renders the transformable spinal fixation device relatively rigid compared with the primary phase or material. Upon implantation into the body, the secondary phase or material may be contacted by water or body fluids, however, and become more flexible. The transformable spinal fixation device thereby also becomes more flexible.

The primary phase or material of the transformable spinal fixation device may be any of the materials or compositions mentioned above in reference to the transformable implantable device. For example, the primary phase or material may be a metal, ceramic, polymer, or composite thereof. The secondary phase or material likewise may be any of the materials or compositions mentioned above in reference to the transformable implantable device. In a preferred embodiment, the secondary phase or material is a hydrophilic polymer or hydrogel. In a more preferred embodiment, the hydrophilic polymer or hydrogel is bio-absorbable such that it is absorbed and removed by the body following implantation. Additionally, the transformable spinal fixation implant may include advantageous additives or agents such as growth factors, antibiotics, immunosuppressants, narcotics, muscle relaxers, nutrients, and any of the other additives and agents discussed previously with reference to the transformable implant.

The transformable spinal fixation implant preferably comprises a transformable stabilizing element fabricated from the primary and secondary phases or materials by solution casting, dispersion dipping, extrusion, injection molding, compression molding, bonding, laminating, or otherwise. The transformable stabilizing element acts to stabilize the vertebrae to which it is anchored by associated anchoring means. A preferred form of the transformable stabilizing element is a bundle of fibers like a tether, cord, cable, band, or tape produced by braiding, weaving, knitting, or sewing fibers of the primary phase or material.

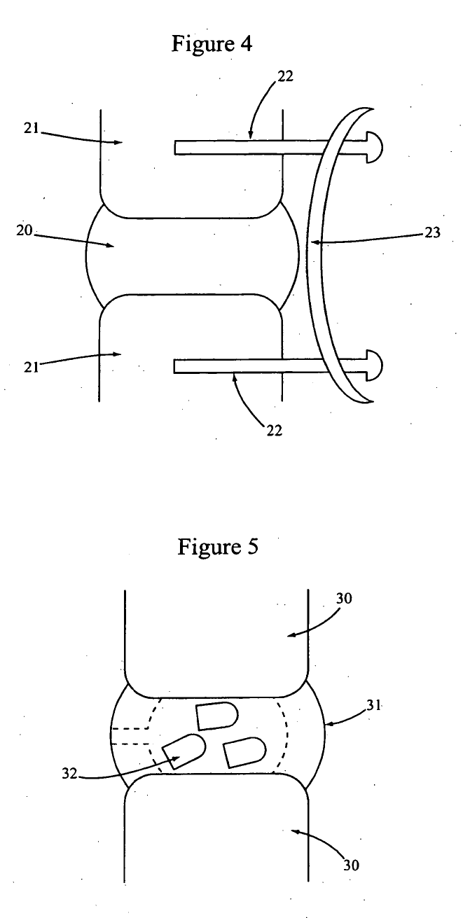

Another exemplary form of the transformable stabilizing element, as illustrated in FIG. 4, is a transformable rod 23 or rods that extend substantially parallel to the vertebral column. The transformable rod or rods may be of any appropriate cross sectional geometry including, but not limited to, circular, ovate, square, rectilinear, hexagonal, and octagonal. The transformable rod is preferably attached to adjacent vertebral bodes 21 by anchoring means 22 and spans the vertebral joint 20, providing support to the joint. Though FIG. 4 illustrates the transformable stabilizing element spanning only one vertebral joint, it is understood that the transformable stabilizing element may span more than one vertebral joint if desired. The transformable rod 23 is initially relatively rigid compared to the primary phase or material. Upon implantation, however, the transformable rod 23 may be contacted by water or body fluids and become more flexible.

FIG. 3 is another exemplary illustration of a transformable rod-like stabilizing element 13. The transformable stabilizing element 13 may include a hydrogel-filled core 14 or other elastic material that imparts an extra measure of elasticity to the transformable stabilizing element. Anchoring means 11 attaches the transformable stabilizing element 13 to adjacent vertebral bodies 10. The transformable stabilizing element 13 spans the intervertebral joint 12, providing support to the joint. Though FIG. 3 illustrates the transformable stabilizing element spanning only one vertebral joint, it is understood that the transformable rod-like stabilizing element may span more than one vertebral joint if desired. The transformable stabilizing element 13 is initially relatively rigid compared to the primary phase or material. Upon implantation, however, the transformable stabilizing element 13 may be contacted by water or body fluids and become more flexible.

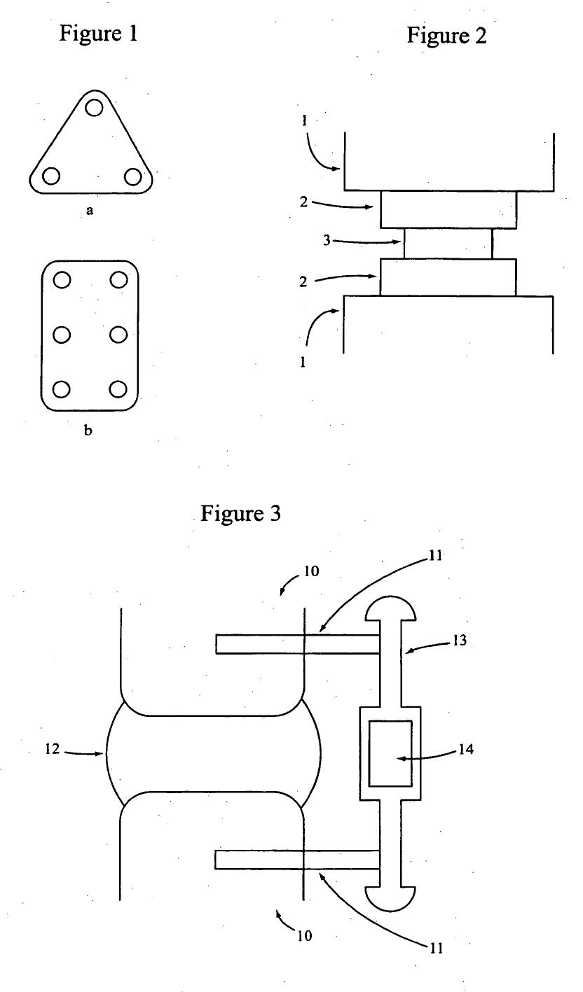

Still another example of the transformable stabilizing element, as illustrated in FIG. 1, embodiments a and b, is a pyramidal, triangular, or rectangular plate that may be attached to one or more vertebrae. FIG. 7 illustrates an exemplary installation of the transformable stabilizing element configured as a plate. The transformable stabilizing element 51 is attached to adjacent vertebrae 52 by anchoring means 50 and spans the intervertebral disc joint 53. The transformable stabilizing element 51 is initially relatively rigid compared to the primary phase or material. Upon implantation, however, the transformable stabilizing element 51 may be contacted by water or body fluids and become more flexible.

One skilled in the art will appreciate the myriad configurations that the transformable spinal fixation implant may take.

The anchoring means used to attach the transformable stabilizing element to the vertebrae that are to be fixed may be in any one of numerous forms or combinations thereof. For example, screws, studs, bolts, staples, sutures, and tacks are examples of the anchoring means that may be used to attach the transformable stabilizing element to the vertebrae. In a preferred embodiment, the anchoring means includes an upper shank portion and a lower threaded portion having a screw thread. The lower portion is cooperatively connected to the shank portion. The screw thread has segmented areas, wherein a rotary force may be applied to the shank portion whereby the threaded portion is driven into and secured into the pedicles or other portions of the vertebral body. The screw thread may have segmented areas, wherein after a period of time the vertebra's bony regrowth encompasses the segmented areas to further secure the threaded portion to the pedicles.

The transformable spinal fixation device may be positioned in numerous configurations about the vertebral column. For example, the transformable stabilizing element may be placed on the anterior face of the vertebrae. In another example, the transformable stabilizing element is mounted on the sides of the vertebrae. Alternatively, the transformable stabilizing element may be anchored to different surfaces of different vertebrae, for example the anterior face of a lower vertebrae and the side of a higher vertebrae. Also, more than one transformable stabilizing element may be concurrently anchored to the vertebrae. One skilled in the art will again appreciate the many different configurations in which the transformable spinal fixation device may be positioned with respect to the vertebral column.

In a preferred embodiment, the transformable spinal fixation device is relatively flexible until substantial dehydration of the secondary phase or material is effected by freeze-drying, air-drying, heating, vacuum-drying, or otherwise. Substantial dehydration of the secondary phase or material makes the transformable device relatively rigid. The relative rigidity of the transformable spinal fixation device may facilitate implantation of the device. Additionally, the relative rigidity of the transformable device may, during healing, additionally support the stresses placed upon the vertebral column. As the secondary phase or material is substantially re-hydrated by body fluids, the transformable spinal fixation device will again resume its relatively flexible state. The period of re-hydration may be adjusted by affecting the permeability of the transformable fixation device. The less permeable to water the transformable fixation device is, the longer the period of rigidity. Again, this may be advantageously employed to ensure that the transformable fixation device provides adequate support while the vertebral column is still healing.

In another exemplary embodiment, a transformable implantable device in the form of an transformable intervertebral disc implant device is provided comprising at least one primary phase or material and a secondary phase or material that is relatively rigid when compared to the primary phase or material. The secondary phase or material renders the transformable intervertebral disc implant device relatively rigid compared to the primary phase or material. Upon implantation into the body, the secondary phase or material may be contacted by water or body fluids and become more flexible. The transformable intervertebral disc implant device thereby also becomes more flexible.

The primary phase or material of the transformable intervertebral disc implant may be any of the materials or compositions mentioned above in reference to the transformable implantable device. For example, the primary phase or material may be a metal, ceramic, polymer, or composite thereof. The secondary phase or material likewise may be any of the materials or compositions mentioned above in reference to the transformable implantable device. In a preferred embodiment, the secondary phase or material is a hydrophilic polymer or hydrogel. In a more preferred embodiment, the hydrophilic polymer or hydrogel is bio-absorbable such that it is absorbed and removed by the body following implantation. Additionally, the transformable intervertebral disc implant may include advantageous additives or agents such as growth factors, antibiotics, immunosuppressants, narcotics, muscle relaxers, nutrients, or any of the other additive and agents discussed previously with reference to the transformable implant.

The transformable disc implant may be fabricated in any number of different configurations, as will be appreciated by one skilled in the art. For example, as illustrated in FIG. 5, the transformable implant may be in the form of transformable “bullets” or short rods 32 that may be inserted into the disk or nucleus space 31 (partially evacuated, fully evacuated, or not evacuated). Implantation may be facilitated by the use of, for example, a cannula by which the transformable bullets are inserted into the disc or nucleus space. Before insertion, the transformable bullets 32 are relatively rigid, when compared to the primary phase or material. Once inserted, the transformable bullets 32 may be contacted with water or body fluids, may expand to fill the disc or nucleus space 31, and/or become more flexible.

In another exemplary transformable disc implant, as illustrated in FIG. 2, the primary and secondary phases or materials are used to fabricate a transformable core 3 that is joined to two rigid outer members 2. The rigid outer members may, for example, be fabricated from a biocompatible metal such as stainless steel, particularly 316L stainless steel, cobalt-chrome alloys, cobalt-nickel-chrome alloys, particularly MP35N, titanium, titanium alloys, particularly Ti-6A1-4V, nickel-titanium shape memory alloys, composites thereof, and the myriad different grades of these metals. The transformable implant preferably is surgically placed in the disc space between adjacent vertebrae 1, thereby providing support to the vertebral joint and absorbing at least a portion of the compressive forces of the vertebral column. The transformable core 3 is initially relatively rigid, when compared to the primary phase or material. Upon contact with water or body fluids, however, the transformable core becomes more flexible. This may allow the transformable core to provide extra support to the vertebral joint during the early stages of healing when the transformable core is still relatively rigid but also allow an extra degree of mobility after healing has progressed when the transformable core becomes more flexible.

The transformable implant may likewise be in the form of a kidney-shaped disc meant to mimic the natural shape of the intervertebral disc. Another example is an transformable intervertebral implant in a C-shaped configuration. One skilled in the art will recognize the many different configurations the transformable intervertebral disc implant may take.

The transformable intervertebral implant preferably is relatively flexible until substantial dehydration of the secondary phase or material is effected by freeze-drying, air-drying, heating, vacuum-drying, or otherwise. Substantial dehydration of the secondary phase or material makes the transformable device relatively rigid. The relative rigidity of the transformable intervertebral implant may facilitate implantation of the device. Additionally, the relative rigidity of the transformable device may, during healing, additionally support the stresses placed upon the vertebral column. One skilled in the art also will appreciate the many different ways in which the transformable intervertebral implant may be inserted into the evacuated disc or nucleus space. For example, in minimally invasive surgery, the transformable implant may be inserted using a cannula. Alternatively, the transformable implant may be placed by hand in the evacuated disc space. The disc space may be evacuated by curettage, suction, laser nucleotomy, chemonucleolysis, or any other appropriate surgical method.

As the secondary phase or material is substantially re-hydrated by body fluids, the transformable intervertebral implant will again resume its relatively flexible state compared to the primary phase or material. The period of re-hydration may be adjusted by affecting the permeability of the transformable intervertebral device. The less permeable to water the transformable intervertebral device is, the longer the period of rigidity.

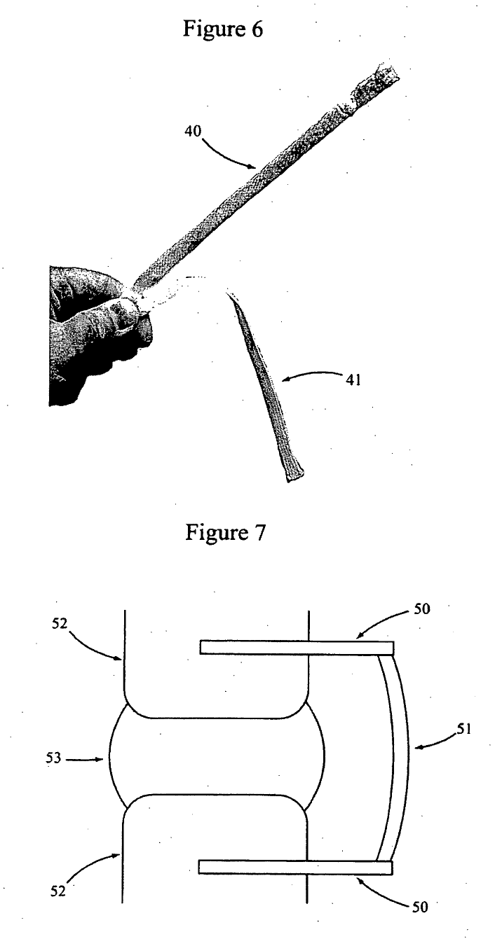

In another exemplary embodiment of the present invention, there is provided a transformable implant in the form of a transformable spinal ligament repair and reinforcement device. FIG. 6 illustrates a preferred transformable spinal ligament repair device in its relatively rigid 40 and more flexible 41 states. The transformable spinal ligament repair device comprises at least one primary phase or material and a secondary phase or material that is relatively rigid compared with the primary phase or material. The secondary phase or material renders the transformable spinal ligament repair device relatively rigid 40 compared to the primary phase or material. Upon implantation into the body, the secondary phase or material may be contacted by water or body fluids and become more flexible. The transformable spinal ligament repair device thereby also becomes more flexible 41. It is understood that the transformable spinal ligament repair and reinforcement device may span one or more intervertebral joints.

The invention now will be described in more detail with reference to the following non-limiting examples.

### EXAMPLE 1

A hollow rod comprised of high modulus polyethylene fibers (SPECTRA® fibers, commercially available from Honeywell International, Inc., Colonial Heights, Va.) was cut in half, and one half was stretched and inserted into the other half to form a two-layer composite rod. The composite rod of polyethylene fibers constitutes the primary phase or material and gelatin will constitute the secondary phase or material. The composite transformable rod was initially relatively flexible. Gelatin was injected into the composite rod until full, the ends of the hollow rod were tied off, excess gelatin wiped off, and the gelatin soaked composite rod was allowed to dry in ambient conditions. Upon drying, the rod was rigid. The rigid composite rod then was re-hydrated it in 37° C. water and the rod eventually became flexible. After a few minutes of re-hydration, the composite rod was still rigid, but as time passed, the composite rod slowly became flexible over a period of about one hour. This composite rod will be useful as a stabilization element in a transformable spinal fixation device. For use as a stabilization element, (the remaining portion of this example is prophetic) and prior to re-hydration (which may occur in vivo), the relatively rigid composite rod will be cut to an appropriate length and, if desired, ground to points at its ends. During insertion into the body, the gelatin will absorb body fluids, thereby lubricating the outside surface of the transformable rods and facilitating insertion. The transformable rods will subsequently be connected by anchoring means, for example pedicle screws or set screws, to the vertebrae that are to be fixed. The gelatin will gradually absorb water or other body fluids and the transformable rods will resume their relatively flexible state. Over time, the gelatin will itself be absorbed by the body, leaving behind the flexible composite rod as a stabilizing element.

### EXAMPLE 2

A transformable anterior tension band is created by casting a gelatin solution into a braided band of polyethylene fibers. The braided band of polyethylene fibers constitutes the primary phase or material and the gelatin solution constitutes the secondary phase or material. The transformable band is initially relatively flexible. Excess gelatin is removed from the surface of the band and the band is substantially dehydrated in a vacuum oven, by heating, or by other means. Upon substantial dehydration, the transformable band will become relatively rigid as the movement of the polyethylene fibers is restricted by the dried gelatin particles embedded in the fibers. During insertion into the body, the gelatin absorbs body fluids, thereby lubricating the outside surface of the transformable band and facilitating insertion. The transformable band is subsequently connected by anchoring means, for example staples or screws, to the vertebrae. By tensioning the vertebrae, stress shielding effects are avoided because the vertebrae are placed under constant stress. A traditional spinal fixation device is then installed on the same vertebrae to restrict vertebral movement. The gelatin gradually absorbs water from the body fluids and the transformable band resumes its relatively flexible state. Over time, the gelatin is itself absorbed by the body, leaving behind the braided tether as a tensioning element.

### EXAMPLE 3

A transformable intervertebral disc implant is created by casting a gelatin solution into a braided tether of polyethylene fibers. The braided band of polyethylene fibers constitutes the primary phase or material and the gelatin solution constitutes the secondary phase or material. Excess gelatin is removed from the surface of the tether and the tether is substantially dehydrated in a vacuum oven, by heating, or other means. Upon substantial dehydration, the transformable tether becomes relatively rigid as the movement of the polyethylene fiber is restricted by the xerogel particles embedded in the fibers. The tether is cut into short segments that are inserted, for example, by cannula into the disc or nucleus space. The gelatin is rehydrated by the body fluids in the disc or nucleus space and the transformable segments resume the relatively flexible state of the braided tether.

The invention has been described with reference to the non-limiting examples and particularly preferred embodiments. Those skilled in the art will appreciate that various modifications may be made to the invention without departing significantly from the spirit and scope thereof.

What is claimed is:

1. A transformable implantable device comprising:
at least one flexible primary phase or material and a secondary phase or material that is relatively rigid, when compared to the primary phase or material;
wherein the secondary phase or material renders the transformable implantable device relatively rigid compared to the primary phase or material; and
wherein the secondary phase or material, upon implantation, becomes more flexible, thereby rendering the transformable implantable device more flexible.

2. The device of claim 1, wherein the secondary phase or material, before implantation, is in a substantially dehydrated state.

3. The device of claim 1, wherein the secondary phase or material, upon implantation, becomes more flexible because of partial, substantial, or complete rehydration of the secondary phase or material.

4. The device of claim 3, wherein the secondary phase or material is partially, substantially, or completely rehydrated by contact with bodily or surgical fluids.

5. The device of claim 1, wherein the primary phase or material is selected from the group consisting of ceramics, metals, polymers, and composites and mixtures thereof.

6. The device of claim 1, wherein the primary phase or material is a fiber.

7. The device of claim 6, wherein the fiber is selected from the group consisting of polyethylene fibers, polyester fibers, polyaryletherketone fibers, stainless steel filaments, shape-memory metal alloy filaments, and combinations and mixtures thereof.

8. The device of claim 6, wherein the fibers are braided, woven, or non-woven tether, cord, band, or tubing.

9. The device of claim 6, wherein the fiber comprises a braid of fibers of polyethylene and polyester.

10. The device of claim 6, wherein the fiber comprises a braid of fibers of polyethylene and polyaryletherketone.

11. The device of claim 6, wherein the fiber comprises a braid of fibers of polyethylene and stainless steel filaments.

12. The device of claim 6, wherein the fiber comprises a braid of fibers of polyethylene and shape-memory metal alloy filaments.

13. The device of claim 1, wherein the primary phase or material is manufactured by braiding, weaving, knitting, sewing, extrusion, injection molding, compression molding, casting, bonding, or laminating.

14. The device of claim 1, manufactured by solution casting, dispersion dipping, extrusion, injection molding, compression molding, bonding, laminating or machining.

15. The device of claim 1, wherein the secondary phase or material is a polymer or ceramic.

16. The device of claim 15, wherein the polymer is a hydrophilic polymer or hydrogel.

17. The device of claim 16, wherein the hydrophilic polymer or hydrogel is selected from the group consisting of polyethylene oxide, polyethylene glycol, polyvinyl alcohol, polyacrylic acid, polyacrylamide, cellulose, collagen, polysaccharides, gelatin, elastin, silk, keratin, albumin, and copolymers, blends, composites, and laminates thereof.

18. A transformable spinal fixation device comprising:
at least one flexible primary phase or material and a secondary phase or material that is relatively rigid, when compared to the primary phase or material;
wherein the secondary phase or material renders the transformable spinal fixation device relatively rigid compared to the primary phase or material; and
wherein the secondary phase or material, upon implantation, becomes more flexible, thereby rendering the transformable spinal fixation device more flexible.

19. The device of claim 1, wherein the secondary phase or material, before implantation, is in a substantially dehydrated state.

20. The device of claim 18, wherein the secondary phase or material, upon implantation, becomes more flexible because of partial, substantial, or complete rehydration of the secondary phase or material.

21. The device of claim 20, wherein the secondary phase or material is partially, substantially, or completely rehydrated by contact with bodily or surgical fluids.

22. The device of claim 18, wherein the primary and secondary phases or materials form a stabilizing element that is secured to two or more vertebrae by anchoring means.

23. The device of claim 22, wherein the stabilizing element is in the form of a pyramidal, triangular, ovular, square, rectangular, circular, or irregularly shaped plate.

24. The device of claim 22, wherein the stabilizing element is in the form of a elongated rod.

25. A transformable intervertebral disc implant device comprising:
at least one flexible primary phase or material and a secondary phase or material that is relatively rigid, when compared to the primary phase or material;
wherein the secondary phase or material renders the transformable intervertebral disc implant device relatively rigid compared to the primary phase or material; and
wherein the secondary phase or material, upon implantation, becomes more flexible, thereby rendering the transformable intervertebral disc implant device more flexible.

26. The device of claim 1, wherein the secondary phase or material, before implantation, is in a substantially dehydrated state.

27. The device of claim 25, wherein the secondary phase or material, upon implantation, becomes more flexible because of partial, substantial, or complete rehydration of the secondary phase or material.

28. The device of claim 27, wherein the secondary phase or material is partially, substantially, or completely rehydrated by contact with bodily or surgical fluids.

29. The device of claim 25, wherein the transformable intervertebral disc implant is used to replace the nucleus of an intervertebral disc, replace a portion of or entire intervertebral disc, or maintain or increase the interspinous spacing of an intervertebral disc.

30. The device of claim 25, wherein the transformable intervertebral disc implant is in the form of a disc-like shape and additionally comprises two end plates disposed on opposite faces of the disc.

31. The device of claim 30, wherein the end plates comprise a metal chosen from the group consisting of stainless steel, 316L stainless steel, cobalt-chrome alloys, cobalt-nickel-chrome alloys, MP35N cobalt-nickel-chrome alloy, titanium, titanium alloys, Ti-6A1-4V titanium alloy, nickel-titanium shape memory alloys, and composites or mixtures thereof.

32. The device of claim 25, wherein the transformable intervertebral disc implant device is in the form of short rods.

33. A transformable spinal ligament repair and reinforcement device comprising:
at least one flexible primary phase or material and a secondary phase or material that is relatively rigid, when compared to the primary phase or material;
wherein the secondary phase or material renders the transformable spinal ligament repair and reinforcement device relatively rigid compared to the primary phase or material; and
wherein the secondary phase or material, upon implantation, becomes more flexible, thereby rendering the transformable spinal ligament repair and reinforcement device more flexible.

34. The device of claim 1, wherein the secondary phase or material, before implantation, is in a substantially dehydrated state.

35. The device of claim 33, wherein the secondary phase or material, upon implantation, becomes more flexible because of partial, substantial, or complete rehydration of the secondary phase or material.

36. The device of claim 33, wherein the secondary phase or material is partially, substantially, or completely rehydrated by contact with bodily or surgical fluids.

37. The device of claim 33, wherein the primary and secondary phases or materials are in the form of a tether, cord, band, rope, chain, or tube.

38. The device of claim 37, wherein the tether, cord, band, rope, chain, or tube is affixed to two or more vertebrae by anchoring means.
